(19) United States
(12) Patent Application Publication
(10) Pub. No.: US 2007/0299303 A1
(43) Pub. Date: Dec. 27, 2007

(54) FLUID ACTUATOR AND ENDOSCOPE

(75) Inventor: Shinya OGIKUBO, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 11/769,025

(22) Filed: Jun. 27, 2007

(30) Foreign Application Priority Data
Jun. 27, 2006 (JP) 2006-176538

(51) Int. Cl. A61B 1/00

(52) U.S. Cl. 600/102

(57) ABSTRACT

A fluid actuator incorporated in a bending section of an electronic endoscope has two fluid chambers and a connecting channel connecting therebetween. The fluid chambers and the connecting channel are filled with a fluid. The fluid chamber includes a first surface and a second surface opposed to the first surface and to which the connecting channel is connected. The first surface is formed of a stretchy elastic member. A pair of electrodes which elastically deforms in accordance with elastic deformation of the elastic member is attached to the first and second surfaces. Using electrostatic force, generated by the input voltage from a variable power supply, across the electrodes on each fluid chamber, the volumes of the fluid chambers are changed. Thus, the bending section is bent.

### BACKGROUND OF THE INVENTION

1. Field of the Invention

The present invention relates to a fluid actuator suitable for bending a bending section of an endoscope, and the endoscope.

2. Description Related to the Prior Art

Medical diagnoses using electronic endoscopes are prevalent in the medical field. The electronic endoscope has an inserting section inserted into a body of a patient. A front end section of the inserting section incorporates a solid state imaging device such as a CCD. An image signal obtained with the solid state imaging device is subjected to a signal processing in a processor. Thus, an image of an observation target in the body is observed on a monitor.

The inserting section is provided with a bending section for directing the front end section toward a desired direction in the body. Conventional electronic endoscopes bend the bending section by pushing and pulling wires running through the inserting section with the operation of an angle knob provided in an operating section connected to a base portion of the inserting section.

Other than the above conventional method using the wires, various methods are devised to bend the bending section. For example, an endoscope having a fluid actuator in a bending section is suggested (see Japanese Patent Laid-Open Publication No. 05-015485). The fluid actuator is constituted of a flat elastic body having a pressurizing chamber therein, a fiber fitted to the elastic body for restricting a direction of the expansion of the elastic body, and a pressurized tube for feeding the fluid and connected to the pressurizing chamber.

Further, Japanese Patent Laid-Open Publication No. 05-211989 discloses an insertion section of an endoscope in which an elastic actuator and a wire for bending operation are provided through a flexible tube. The elastic actuator expands and contracts in an axial direction with the use of fluid pressure, and the wire transmits the expansion and contraction of the elastic actuator and bends a bending tube. The part of the flexible tube above the elastic actuator has an external skin formed of a soft resin so as to achieve higher flexibility than the other parts.

The conventional methods using the wires require a certain degree of skill to operate the angle knob and bend the bending section to a desired direction. Therefore, an unskilled operator may put a heavy physical burden on the patient by taking a long time to bend the bending section to a desired direction, resulting in a long diagnosing time, or by improper operation of the angle knob such as accidentally bumping the tip of the endoscope against an interior wall of a body part. Moreover, the operation of the angle knob is difficult when the endoscope is inserted into a highly curved lumen such as a large intestine or a small intestine, because bending stress caused by the bending of the inserting section interferes the pushing and pulling of the wires.

Further, the inventions disclosed in the above prior art require a pressurizing/depressurizing mechanism such as a pump or compressor for feeding the fluid to the actuator, resulting in upsizing the diagnosing apparatus.

### SUMMARY OF THE INVENTION

A main object of the present invention is to provide a fluid actuator capable of easily bending a bending section of an endoscope or the like with a simple configuration.

Another object of the present invention is to provide an endoscope capable of performing diagnoses smoothly regardless of experience or skill in operating the endoscope.

To achieve the above and other objects, a fluid actuator according to the present invention includes plural fluid chambers, a connecting channel to connect the fluid chambers, and a pressure generating section. At least a part of each of the fluid chambers is formed of an elastic member. The fluid chambers and the connecting channel are filled with a fluid. The pressure generating section generates pressure for elastically deforming the elastic member so as to move the fluid between the fluid chambers through the connecting channel and to change volumes of the fluid chambers.

It is preferable that the fluid actuator includes a pressure adjusting section for adjusting the pressure. It is preferable that the pressure generating section includes a pair of electrodes attached to an outer surface of the fluid chamber, and elastically deforming in accordance with the elastic deformation of the elastic member, and a power supply for supplying a voltage to the pair of electrodes. In this case, it is preferable that one of the electrodes is attached to a surface of the fluid chamber to which the connecting channel is connected, and the other of the electrodes is attached to a surface opposed to the surface to which the connecting channel is connected. It is preferable that the fluid chamber is formed such that a distance between the electrodes becomes smaller as the pair of electrodes is away from the connecting channel.

Instead, it is also preferable that the pressure generating section includes plural piezoelectric elements, each of which has a pair of electrodes sandwiching a piezoelectric body, disposed on the outer surface of the fluid chamber, and a power supply for supplying a voltage to the pair of electrodes. In this case, it is preferable that the piezoelectric elements are disposed on the surface of the fluid chamber to which the connecting channel is connected, and the opposing surface.

Instead, it is also preferable that the pressure generating section includes a conductive polymer actuator attached to an outer surface of the fluid chamber, and a power supply for supplying a voltage to the conductive polymer actuator. It is preferable that the conductive polymer actuator is disposed on a surface opposed to the surface of the fluid chamber to which the connecting channel is connected. Instead, it is preferable that the conductive polymer actuator is disposed on approximately entire surface of the fluid chamber except for an area connected to the connecting channel.

It is preferable that the pressure adjusting section adjusts the pressure by changing the voltage.

It is preferable that the plural fluid chambers have the same volume in a state of equilibrium. Further, it is preferable that the cross section of each of the fluid chambers has an approximately rectangular shape. It is preferable that the connecting channel connects the plural fluid chambers at equal distances.

It is preferable that the pressure generating section generates pressure between the surface of the fluid chamber to which the connecting channel is connected and the opposing surface. It is preferable that the surface opposed to the surface of the fluid chamber to which the connecting channel is connected is formed the elastic member. It is preferable that the connecting channel is formed of a hard material which is not elastically deformed.

It is preferable that the fluid is one of normal saline solution, water, air, nitrogen, and a rare gas. It is preferable that the elastic member is one of silicone rubber, polyurethane rubber, and latex rubber.

The present invention also includes an endoscope which is inserted into an object to be inspected. The endoscope has a bending section in an inserting section which is inserted into the object. The bending section incorporates a fluid actuator according to claim 1.

It is preferable that the endoscope includes an operating section for operating the bending section, and a drive control section for controlling a drive of the fluid actuator in accordance with operation of the operating section. It is preferable that a forceps channel, an air/water supplying tube, and a cable are inserted through a cavity between the fluid chamber and the connecting channel. It is preferable that the endoscope is provided with a plurality of the bending sections, and the fluid actuator is incorporated in each of the bending sections.

According to the present invention, the fluid actuator includes the plural fluid chambers, the connecting channel to connect the fluid chambers, and the pressure generating section. At least a part of each fluid chamber is formed of the elastic member. The fluid chambers and the connecting channel are filled with the fluid. The pressure generating section generates pressure for moving the fluid between the fluid chambers through the connecting channel, and elastically deforming the elastic member, and changing the volumes of the fluid chambers. Thus, the bending section of the endoscope or the like is easily bent with a simple configuration.

According to the endoscope of the present invention, since the fluid actuator of the claim 1 is incorporated in the bending section, diagnoses are smoothly performed regardless of experience or skill in operating the endoscope.

### BRIEF DESCRIPTION OF THE DRAWINGS

For more complete understanding of the present invention, and the advantage thereof, reference is now made to the following descriptions taken in conjunction with the accompanying drawings, in which:

FIG. 1 is a schematic view showing a configuration of an electronic endoscope system;

FIG. 2 is a block diagram showing a configuration of an electronic endoscope system;

FIG. 3 is an axial section view of a fluid actuator;

FIG. 4 is an axial section view of the fluid actuator;

FIG. 5 is an enlarged cross section view of the fluid actuator in a state that the bending section is bent;

FIG. 6 is an enlarged cross section view of a fluid actuator according to another embodiment;

FIG. 7 is an enlarged cross section view of an example of an arrangement of the fluid actuator;

FIG. 8 shows examples of arrangements of the fluid actuators: wherein in FIG. 8A, two bending sections are bent by the same bending amount in the same direction; in FIG. 8B, two bending sections are bent by different bending amounts in different directions; and in FIG. 8C, one of the two bending sections is bent up and down and the other is bent right and left;

FIG. 9 is an enlarged axial section view of a fluid actuator using piezoelectric elements;

FIG. 10 is an enlarged axial section view of a fluid actuator using conductive polymer actuators;

FIG. 11 is an enlarged cross section view of the fluid actuator shown in FIG. 10;

FIG. 12 is an enlarged axial section view of a fluid actuator using conductive polymer actuators according to another embodiment; and

FIG. 13 is an enlarged cross section view of the fluid actuator shown in FIG. 12.

### DESCRIPTION OF THE PREFERRED EMBODIMENTS

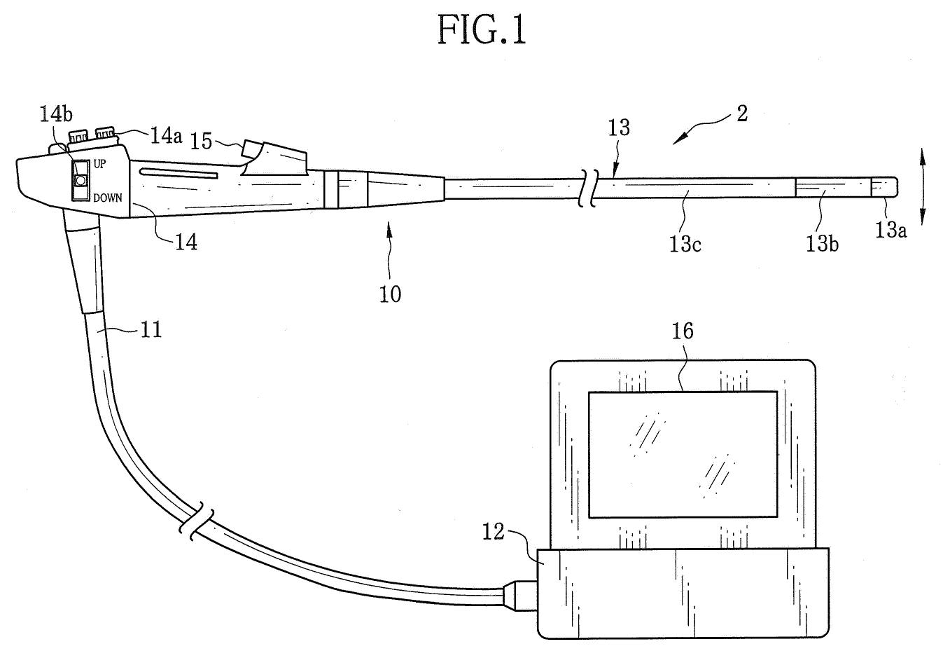

In FIG. 1, an electronic endoscope system 2 is constituted of an electronic endoscope 10, and a processor 12 connected to the electronic endoscope 10 through a cord 11. The electronic endoscope 10 has an inserting section 13 inserted into a body, and an operating section 14 connected to a base portion of the inserting section 13.

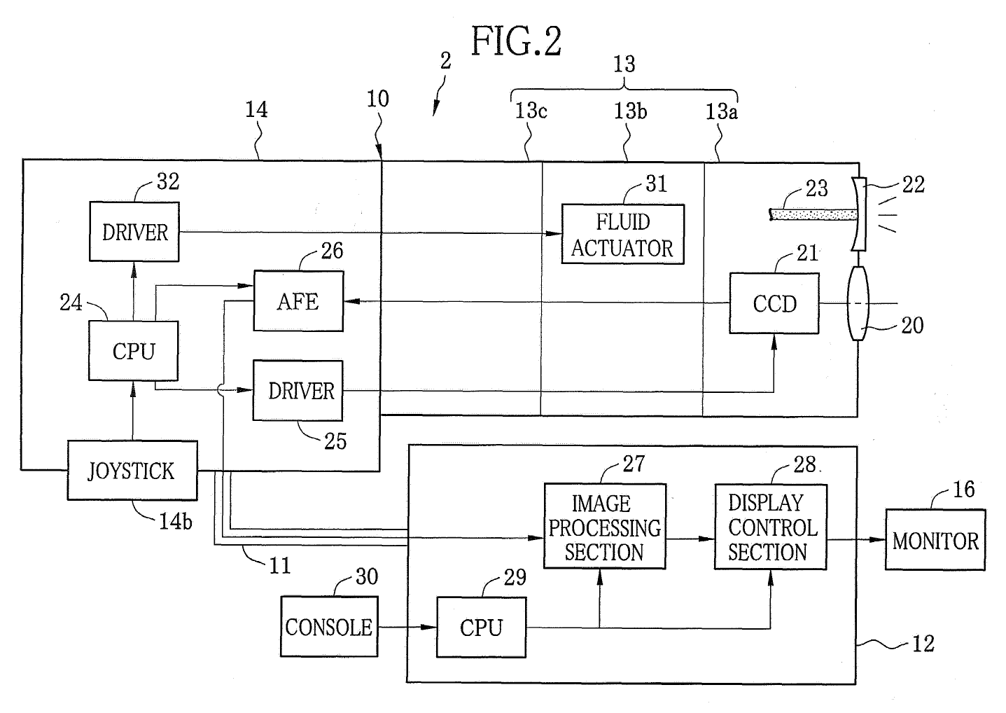

A front end section 13a with an outer diameter of, for example, 12.8 mm is connected to a front end of the inserting section 13. The front end section 13a has an objective optical system 20 for taking in image light of an observation target in the body, a CCD 21 for capturing the image light, and a light window 22 (see FIG. 2) for emitting illumination light from a lighting device (not shown) to the observation target. The front end section 13a further includes a forceps outlet (not shown) connected to a forceps opening 15, a nozzle (not shown) and the like. Through the nozzle, water and/or air is sprayed to wash an observation window which protects the objective optical system 20. The image in the body is obtained by the CCD 21 and displayed on a monitor 16 of the processor 12.

A bending section 13b is provided at a rear of the front end section 13a. To bend the bending section 13b up or down as indicated by arrows in FIG. 1, a fluid actuator 31 (see FIG. 2) incorporated in the bending section 13b is driven by operating a joystick 14b provided in the operating section 14. Thus, the front end section 13a can be pointed up or down inside the body.

A flexible section 13c is provided at a rear of the bending section 13b. The flexible section 13c is several meters long to reach an observation target, and to keep a certain distance between an operator and a patient so as not to hinder the operation of the operating section 14.

In FIG. 2, an end of a light guide 23 from the lighting device is attached to the light window 22. The light guide 23 is provided through the inserting section 13 and the operating section 14 to transmit the light emitted from the lighting device to the light window 22.

Based on a drive control signal from a driver 25 connected to a CPU 24, the CCD 21 receives the image light of the observation target focused onto an image capture surface through the objective optical system 20, and outputs an image signal corresponding to the image light from each pixel.

Under a control of the CPU 24, an Analog Front End (AFE) 26 performs correlate double sampling, amplification, and A/D conversion to the image signal output from the CCD 21, and converts the image signal into a digital image signal. The digital image signal output from the AFE 26 is sent to an image processing section 27 of the processor 12 through the cord 11.

The image processing section 27 performs digital signal processing such as gradation correction, edge enhancement, and gamma correction to the digital image signal, and generates a digital video signal. A display control section 28 performs various image processing such as mask generation and addition of character information to the digital video signal, and displays the digital video signal as an image on the monitor 16.

A CPU 29 integrally controls the entire operation of the processor 12. To the CPU 29, a console 30 for operation and various settings is connected. The CPU 29 operates each section of the processor 12 in accordance with the operation signal input from the console 30.

The fluid actuator 31 is incorporated in the bending section 13b. To the fluid actuator 31, a driver 32 which is controlled by the CPU 24 is connected. The CPU 24 generates a drive control signal in accordance with the operation signal from the joy stick 14b, and outputs the drive control signal to the driver 32. On the basis of the drive control signal from the CPU 24, the driver 32 drives the fluid actuator 31.

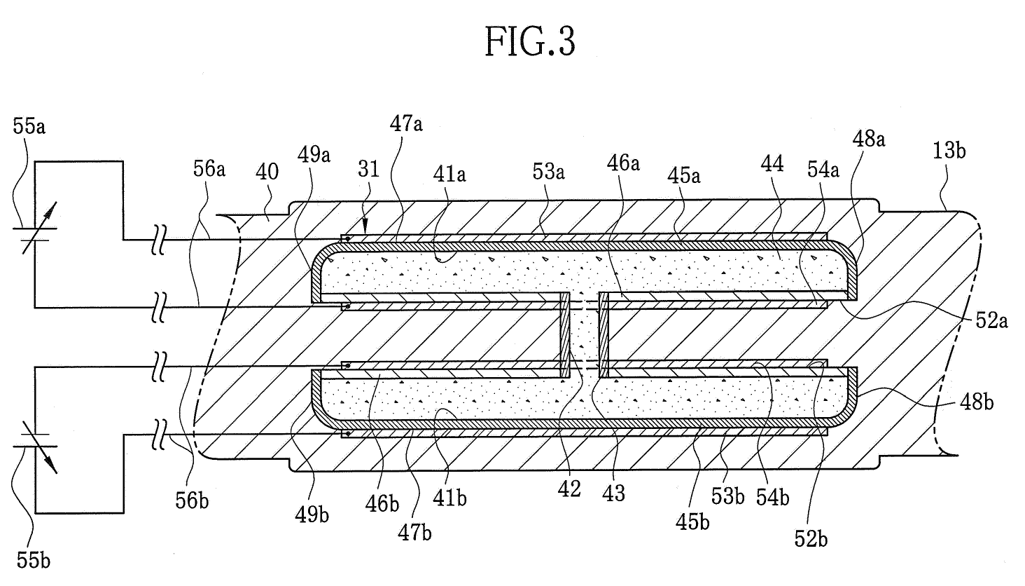

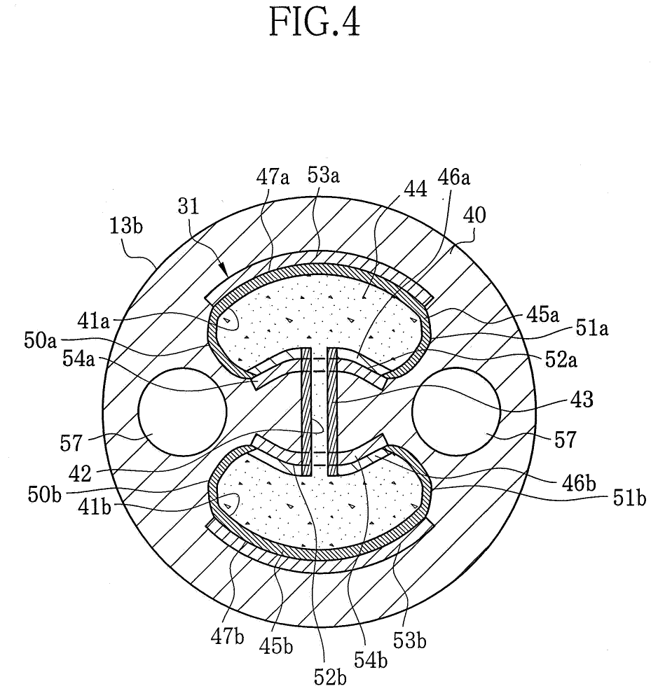

In FIGS. 3 and 4, the bending section 13b is covered with an external tube 40 formed of a stretchy material such as rubber. It is also possible to form a portion of the external tube 40 around the fluid actuator 31 with the stretchy material, and other portions with vinyl or such a flexible, but not stretchy material.

The fluid actuator 31 has two fluid chambers 41a and 41b, and a connecting channel 42 connecting the fluid chambers 41a and 41b. The fluid chambers 41a and 41b are vertically symmetric with respect to an axis of the inserting section 13, and have the same volume in a state of equilibrium shown in FIGS. 3 and 4. Cross sections of the fluid chambers 41a and 41b are approximately rectangular and long in the axial direction of the inserting section 13. Outer peripheral surfaces of the fluid chambers 41a and 41b have the same curve as a perimeter of the inserting section 13.

The connecting channel 42 has an approximately cylindrical shape, and a center thereof in the height direction matches with the axis of the inserting section 13. A connecting member 43 constituting the connecting channel 42 is formed of a hard material which is not elastically deformed, for example, a metal such as aluminum, stainless steel, titanium, or titanium alloy, a carbon fiber, or a resin material such as plastic, with a sufficient thickness to be resistant to deformation.

The fluid chambers 41a and 41b, and the connecting channel 42 are filled with a fluid 44. The fluid 44 is, for example, one of normal saline solution, water, air, nitrogen, and rare gas such as argon, helium, or the like. The fluid 44 moves between the fluid chambers 41a and 41b through the connecting channel 42.

Note that the fluid chambers 41a and 41b have the same configuration. Therefore, hereinafter, only the fluid chamber 41a is described, and the same elements of the fluid chamber 41b have the same numeral, followed by a suffix “b”.

The fluid chamber 41a is constituted of two types of elastic members 45a and 46a. The elastic member 45a is shaped into a first surface 47a of the fluid chamber 41a close to the external tube 40 (that is, a surface of the fluid chamber 41a opposed to a second surface 52a to which the connecting channel 42 is connected), and side surfaces 48a, 49a, 50a, and 51a (see FIG. 4 for 50a and 51a). The side surfaces 48a and 49a are placed opposite in the axial direction of the inserting section 13 and the side surfaces 50a and 51a are placed opposite in the direction vertical to the axial direction. The elastic member 45a is a stretchy material such as, for example, silicone rubber, polyurethane rubber, or latex rubber. The elastic member 46a is shaped into the second surface 52a of the fluid chamber 41a. The elastic member 46a is a flexible but not stretchy material such as, for example, a metal such as aluminum, stainless steel, titanium, or titanium alloy, a carbon fiber, or a resin material such as plastic. The elastic member 46a is made relatively thin so that it will be more flexible.

A pair of electrodes 53a and 54a having approximately the same size is attached to the first surface 47a and the second surface 52a, that is, an upper surface of the elastic member 45a and a lower surface of the elastic member 46a, except for an area of the connecting channel 42. The electrodes 53a and 54a are formed of a stretchy or a flexible material, for example, a polymer material mixed with carbon particles, so that the electrodes 53a and 54a can deform with the elastic members 45a and 46a.

The electrodes 53a and 54a are connected to wires 56a connected to a variable power supply 55a in the driver 32. The variable power supply 55a generates a voltage in accordance with a drive control signal from the CPU 24, and supplies the voltage between the electrodes 53a and 54a. In FIG. 4, a forceps channel, an air/water supply tube, a cable connected to the CCD 21, and the like are inserted through cavities 57 on both sides of the connecting channel 42.

To observe the inside of the body with the above-configured electronic endoscope system 2, the electronic endoscope 10, the processor 12, and the lighting device are turned on, and the inserting section 13 is inserted into the body. Under the light from the light window 22, the images from the CCD 21 are observed on the monitor 16.

The image light from the observation target captured through the objective optical system 20 is focused onto the image capture surface of the CCD 21. Thereby, the image signal is output from the CCD 21. Then, the image signal is converted into the digital image signal in the AFE 26, and input to the image processing section 27 of the processor 12 through the cord 11.

The image processing section 27 performs various signal processing to the digital image signal from the AFE 26, and thereby the digital video signal is generated. The digital video signal is subjected to various image processing in the display control section 28, and displayed as an image on the monitor 16.

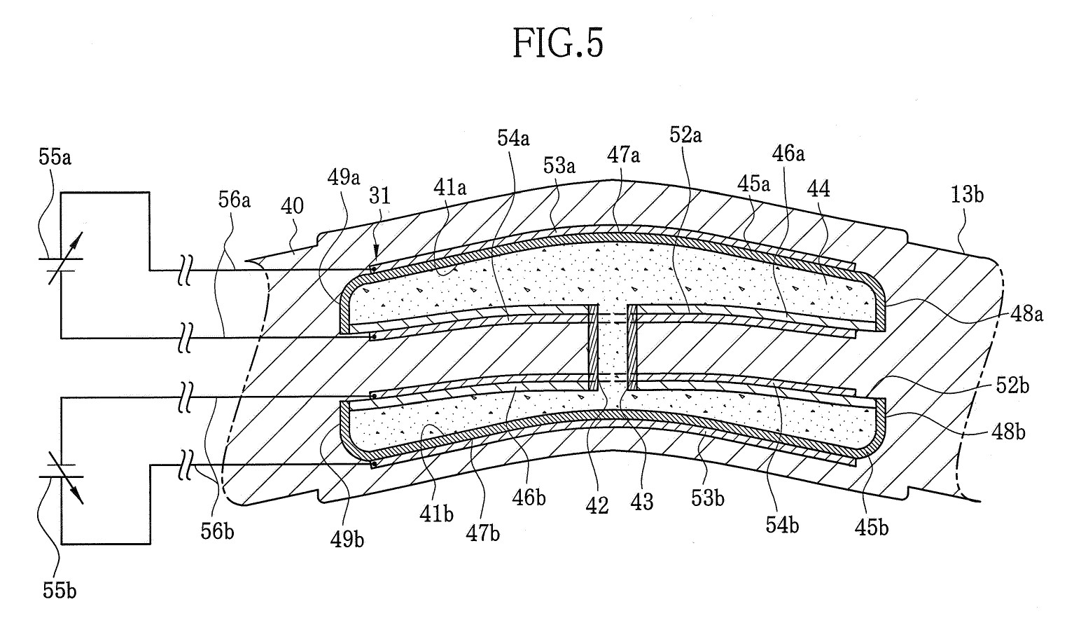

Referring to FIG. 5, the operation of the fluid actuator 31 is described. When the joystick 14b is in the neutral position as shown in FIG. 1, a preset reference voltage is supplied from the variable power supplies 55a and 55b between the electrodes 53a and 54a, and between the electrodes 53b and 54b respectively. The reference voltage is set in a value such that the fluid actuator 31 achieves a state of equilibrium, that is, an electrostatic force between the electrodes 53a and 54a cancels a fluid pressure of the fluid 44 applied to inner walls of the fluid chamber 41a, and an electrostatic force between the electrodes 53b and 54b cancels the fluid pressure of the fluid 44 applied to inner walls of the fluid chamber 41b, as shown in FIGS. 3 and 4. Thereby, the bending section 13b is kept straight and not bent in any directions.

On the other hand, when the joystick 14b is moved to a “down” position, as shown in FIG. 1, to turn the front end section 13a downward, the drive control signal corresponding to the operation signal from the joystick 14b is generated in the CPU 24, and output to the driver 32.

The driver 32 increases the voltage of the variable power supply 55b to a larger value than the reference voltage in accordance with the drive control signal from the CPU 24. The amount of the voltage increase is determined in accordance with the moving amount of the joystick 14b.

If the voltage from the variable power supply 55b becomes larger than the reference voltage, the state of equilibrium shown in FIGS. 3 and 4 is disturbed, and the electrostatic force between the electrodes 53b and 54b becomes larger than the fluid pressure of the fluid 44 applied to the inner walls of the fluid chambers 41a and 41b. Thereby, as shown in FIG. 5, the elastic members 45b and 46b, that is, the first surface 47b and the second surface 52b of the fluid chamber 41b are drawn to each other. As a result, the first surface 47b is elastically deformed such that a center portion thereof is dented, and the second surface 52b bends toward the first surface 47b. In association with the elastic deformation of the first surface 47b and the second surface 52b, a volume of the fluid chamber 41b is reduced, and the fluid 44 is pushed from the fluid chamber 41b to the fluid chamber 41a through the connecting channel 42.

Thereby, due to the fluid pressure of the pushed fluid 44, the elastic member 45a, that is, the first surface 47a is elastically deformed such that the center portion of the first surface 47a curves outward. In addition, the elastic member 46a, that is, the second surface 52a bends toward the fluid chamber 41b. In association with the elastic deformation of the first surface 47a and the second surface 52a, the volume of the fluid chamber 41a increases.

As described above, the volumes of the fluid chambers 41a and 41b are changed, and in accordance with the volume changes, shapes of the fluid chambers 41a and 41b are changed. Accordingly, the bending section 13b bends downward and the front end section 13a points downward. On the other hand, when the joystick 14b is moved to an “up” position shown in FIG. 1, the volume of the fluid chamber 41a is reduced, and the volume of the fluid chamber 41b increases contrary to the above described operation.

As described above, since the electronic endoscope 10 is incorporated with the fluid actuator 31 which bends the bending section 13b by changing the volumes of the fluid chambers 41a and 41b with the use of the electrostatic force between the electrodes 53a and 54a or that between the electrodes 53b and 54b, it becomes easy to bend the bending section 13b to a desired direction compared with the conventional method using the wires. In addition, since the fluid actuator 31 is driven and controlled only by the variable power supplies 55a and 55b, a large apparatus such as a pump, a compressor, or the like becomes unnecessary.

Moreover, it is easy to produce the fluid actuator 31 of a small size with the use of common rubber molding technology. Accordingly, the present invention contributes to reduction of the diameter of the inserting section 13.

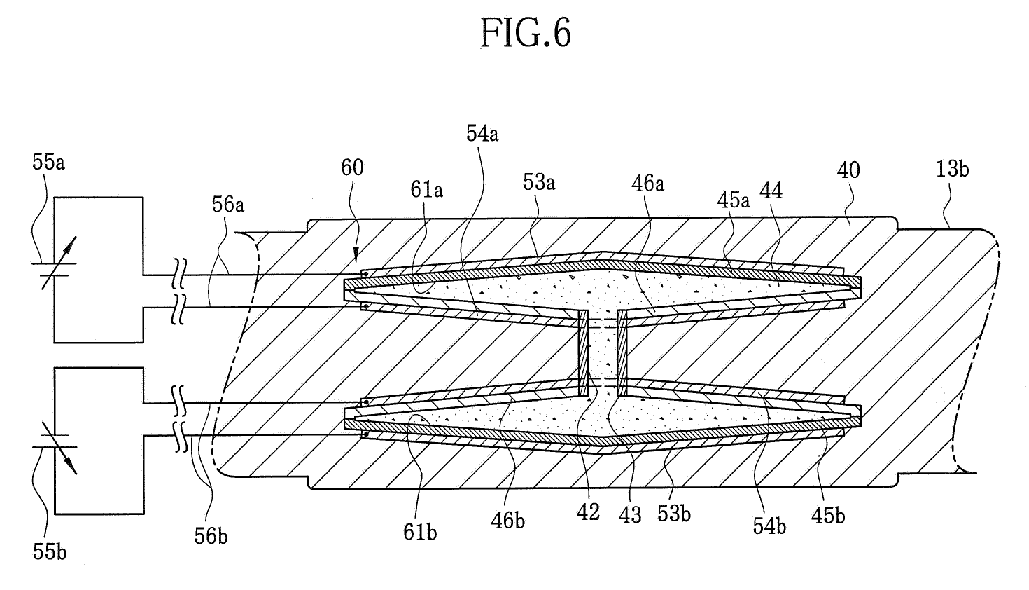

In the above embodiment, the approximately rectangular shaped fluid chambers 41a and 41b are described as an example. It is also possible to use fluid chambers 61a and 61b of a fluid actuator 60 shown in FIG. 6. A distance between the electrodes 53a and 54a, and that between the electrodes 53b and 54b are reduced as the electrodes 53a and 54a, and 53b and 54b are away from the connecting channel 42. Thus, electrostatic force between the electrodes increases toward both end portions of each of the fluid chambers 61a and 61b. As a result, the fluid 44 is squeezed from the end portions to the center portion, and thus the fluid 44 is efficiently pushed.

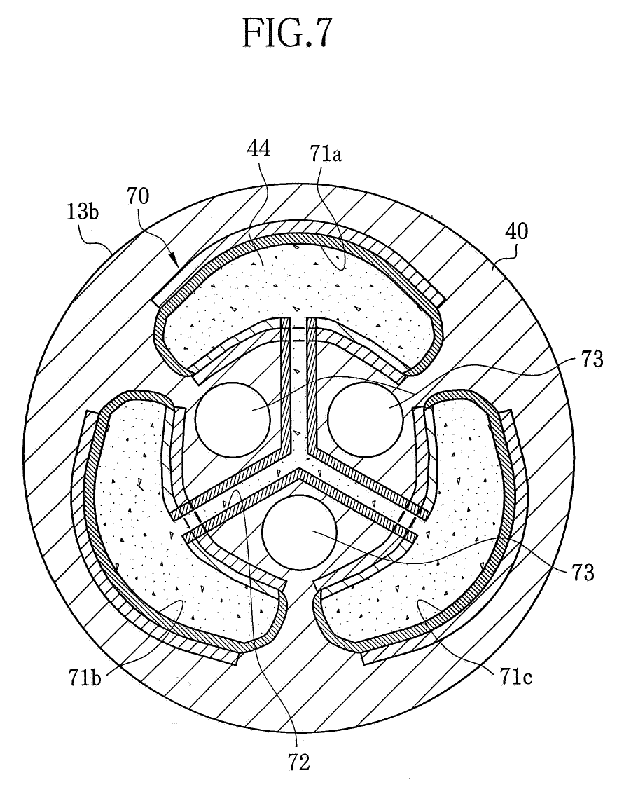

In the first embodiment, the bending section 13b is bent up and down with the use of the fluid actuator 31 having two fluid chambers 41a and 41b as an example. It is also possible to provide more than two fluid chambers to the fluid actuator. For example, it is possible to provide three fluid chambers 71a, 71b, and 71c in a fluid actuator 70 as shown in FIG. 7. The fluid chambers 71a to 71c can be placed every 1200 with respect to a peripheral direction of the external tube 40 so as to make the bending section 13b bendable in three directions.

In this case, a connecting channel 72 connects the fluid chambers 71a, 71b, and 71c at equal distances. Thereby, a fluid pressure is equally transmitted to each fluid chamber when the fluid 44 moves among the fluid chambers 71a, 71b, and 71c. As a result, each fluid chamber can be bent by the same amount in the same drive condition. Note that also in this case, the forceps channel, the air/water supplying tube, the cable connected to the CCD 21, and the like are inserted through cavities 73 between the fluid chambers 71a, 71b or 71c and the connecting channel 72 in the same manner as above.

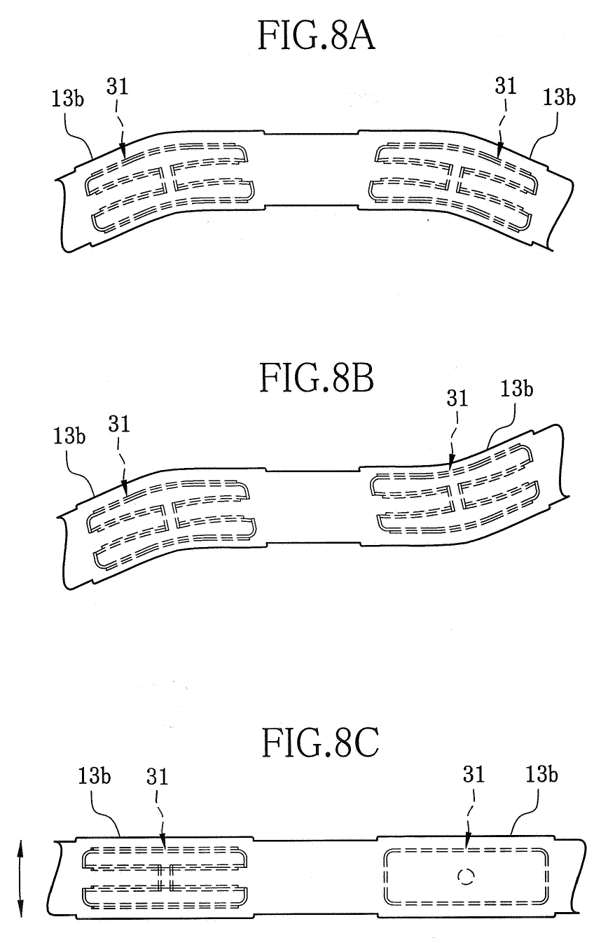

It is also possible to provide plural bending sections 13b along the axial direction of the inserting section 13 instead of the above embodiment having only one bending section 13b. For example, as shown in FIG. 8A, two bending sections 13b can be provided. The electrodes 53a and 54a, and electrodes 53b and 54b of the two fluid actuators 31 incorporated in the bending section 13b are connected by wires. Voltages are supplied from the variable power supplies 55a and 55b between the electrodes 53a and 54a, and between the electrodes 53b and 54b respectively in the same manner as above. Thus, two bending sections 13b are bent in the same direction by the same bending amount. Naturally, the total bending amount is increased compared to the case where only one bending section 13b is provided.

As shown in FIG. 8B, it is also possible to individually control two fluid actuators 31. Thereby, it becomes possible to bend two bending sections 13b by different bending amounts and in different directions. As shown in FIG. 8C, it is also possible to dispose two fluid actuators 31 shifted by 90° with respect to each other in the peripheral direction of the inserting section 13. Thereby, the left fluid actuator 31 is bent up and down as indicated by arrows in FIG. 8B, and the right fluid actuator 31 is bent right and left, vertical to the paper surface of the figure. Thus, the front end section 13a is pointed to up, down, right, and left directions.

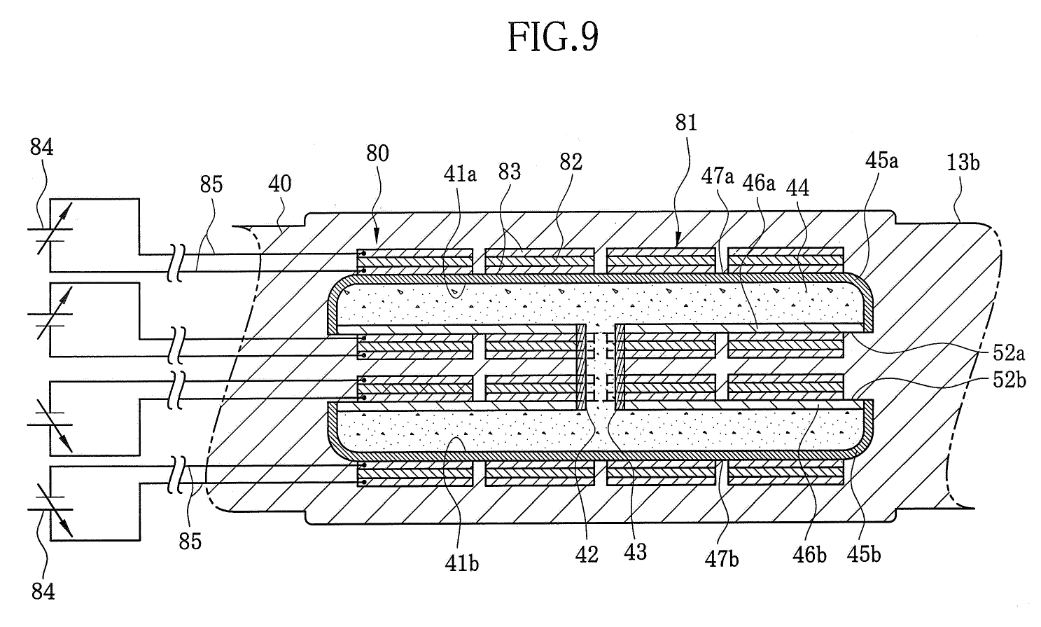

In the above embodiment, a pressure for changing the volume of the fluid chamber is generated with the use of the electrostatic force between the electrodes. However, the present invention is not limited to this. For example, it is also possible to use fluid actuators 80, 90, and 100 shown in FIGS. 9 to 13.

In FIG. 9, the fluid actuator 80 has plural piezoelectric elements 81 arranged in an array on each of the first surface 47a and the second surface 52a of the fluid chamber 41a, and the first surface 47b and the second surface 52b of the fluid chamber 41b. The piezoelectric elements 81 are arranged in the array so as to permit elastic deformation of the elastic member 45a, 45b, 46a, and 46b.

The piezoelectric element 81 is formed of a piezoelectric body 82, and a pair of electrodes 83 sandwiching the piezoelectric body 82. Wires 85 from a variable power supply 84 are connected to the pair of electrodes 83. To drive the fluid actuator 80, a voltage is supplied from the variable power supply 84 between the pair of electrodes 83. The volumes of the fluid chambers 41a and 41b are changed with the use of the expansion and contraction forces generated by piezoelectric effect of the piezoelectric body 82. In FIG. 9, only four piezoelectric elements 81 are connected to the wires 85 and the variable power supplies 84. Actually, each of the piezoelectric elements 81 is provided with the variable power supply 84 and the wires 85.

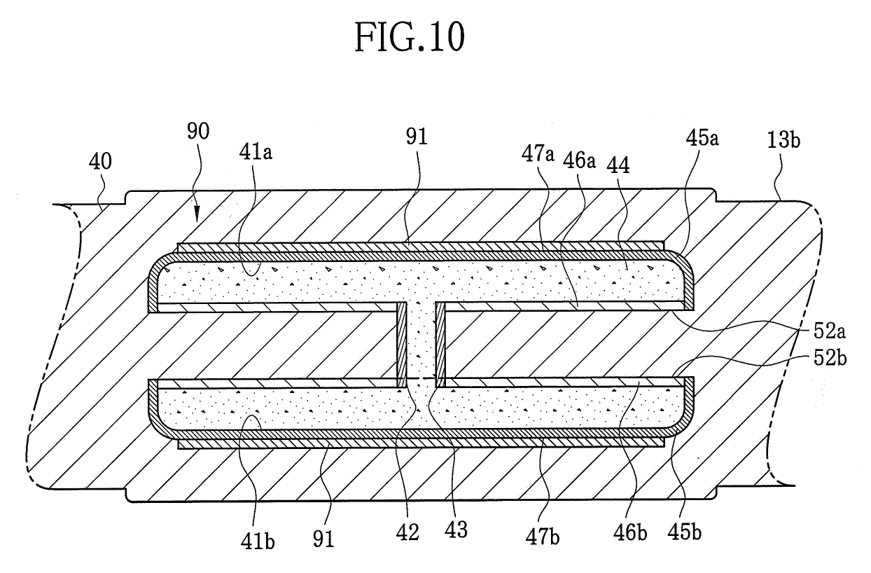

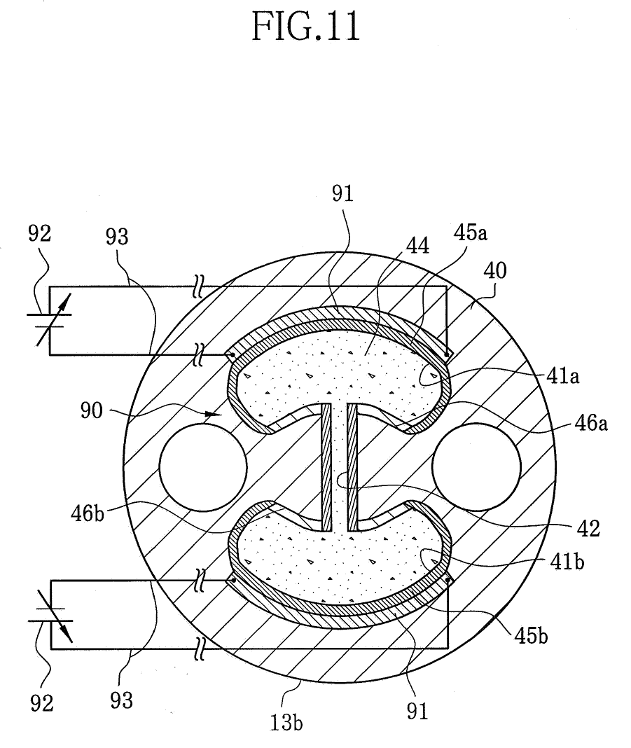

In FIGS. 10 and 11, the fluid actuator 90 has a conductive polymer actuator (hereinafter referred to as a polymer actuator) 91 on each of the first surfaces 47a and 47b of the fluid chambers 41a and 41b. The polymer actuator 91 has a certain degree of elasticity to permit the elastic deformation of the elastic members 45a and 45b.

The polymer actuator 91 is formed of, for example, a polypyrrole film doped with tetrafluoroboric acid or trifluoromethanesulfonic acid. To the both ends of the polymer actuator 91 with respect to the diameter direction of the inserting section 13, wires 93 from the variable power supply 92 are connected. To drive the fluid actuator 90, a voltage is supplied from the variable power supply 92 to the polymer actuator 91. Thereby, expansion and contraction forces are generated by conformation changes in polymer chains caused by input and output of ions. With the use of the expansion and contraction forces, the elastic members 45a and 46a are expanded and contracted, and thus the volumes of the fluid chambers 41a and 41b are changed.

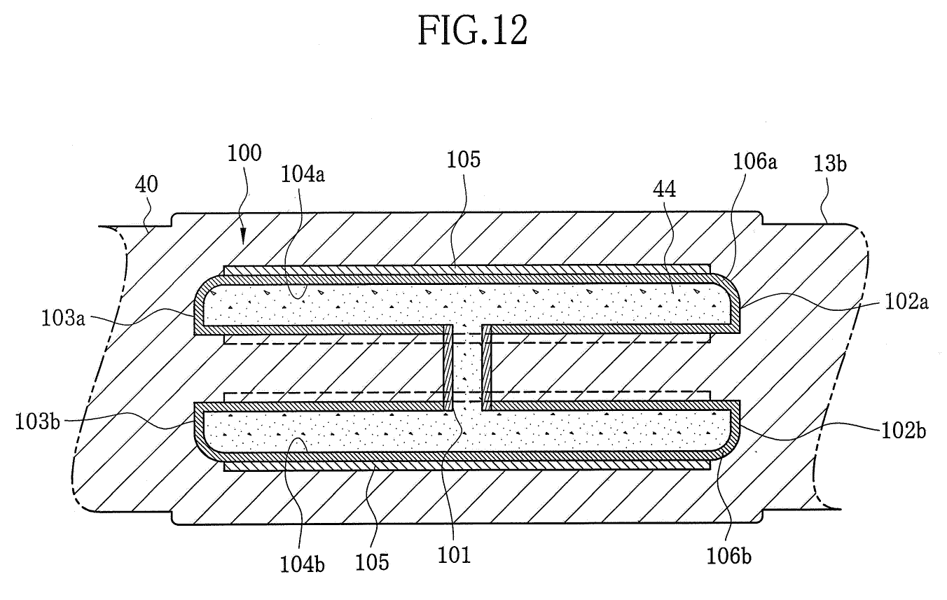

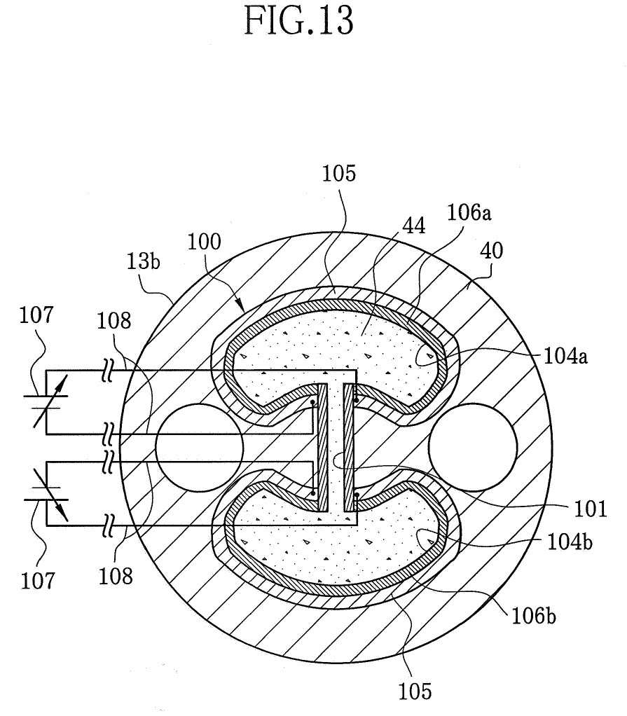

A fluid actuator 100 shown in FIGS. 12 and 13 is constituted of a polymer actuator 105 attached to approximately entire surface of each of fluid chambers 104a and 104b except for an area of the connecting channel 101, an area which is surrounded by two lines extending a width (a diameter) of the connecting channel 101 and both side ends of the fluid chamber in the axial direction, and opposing sides 102a and 103a, and 102b and 103b with respect to the axial direction of the inserting section 13. In this case, the fluid chambers 104a and 104b are solely formed of elastic members 106a and 106b which expand and contract in the same manner as the elastic members 45a and 45b. As with the fluid actuator 90 in FIGS. 10 and 11, wires 108 from a variable power supply 107 are connected to both ends of the polymer actuator 105 with respect to the diameter direction of the inserting section 13. With the use of the expansion and contraction forces generated by the voltage supplied from the variable power supply 107 to the polymer actuators 105, the elastic members 106a and 106b are expanded and contracted, and as a result, the volumes of the fluid chambers 104a and 104b are changed.

The configurations described in the above embodiments are mere examples, and it is possible to properly change the shapes of the fluid chambers, attaching positions of the electrodes, locations of the elastic members, and the like. For example, in the above embodiment, the driver 32 for controlling the drive of the fluid actuator 31 is provided in the operating section 14 of the electronic endoscope 10. However, it is also possible to provide the driver 32 in the processor 12. Further, it is also possible to use other operating member such as an angle knob instead of the joystick 14b.

In the above embodiment, the fluid actuator applied to the electronic endoscope used for medical diagnoses is described as an example. However, the present invention is not limited to the above. The present invention can also be applied to, for example, an ultrasonic endoscope having ultrasonic transducers and the CCD integrally disposed in a front end section, or an inspection scope for inspecting industrial piping.

As described so far, the present invention is not to be limited to the above embodiments, and all matter contained herein is illustrative and does not limit the scope of the present invention. Thus, obvious modifications may be made within the spirit and scope of the appended claims.

What is claimed is:

1. A fluid actuator comprising:
plural fluid chambers, at least a part of each of said fluid chambers being formed of an elastic member;
a connecting channel for connecting said plural fluid chambers, said fluid chambers and said connecting channel being filled with a fluid; and
a pressure generating section for generating pressure for elastically deforming said elastic member so as to move said fluid between said fluid chambers through said connecting channel and to change volumes of said fluid chambers.

2. A fluid actuator according to claim 1 further including:
a pressure adjusting section for adjusting said pressure.

3. A fluid actuator according to claim 1, wherein said pressure generating section includes a pair of electrodes attached to an outer surface of said fluid chamber and elastically deforming in accordance with said elastic deformation of said elastic member, and a power supply for supplying a voltage to said pair of electrodes.

4. A fluid actuator according to claim 3, wherein one of said electrodes is attached to a surface of said fluid chamber to which said connecting channel is connected, and the other of said electrodes is attached to a surface opposed to said surface.

5. A fluid actuator according to claim 4, wherein said fluid chamber is formed such that a distance between said pair of electrodes becomes smaller as said pair of electrodes is away from said connecting channel.

6. A fluid actuator according to claim 1, wherein said pressure generating section includes plural piezoelectric elements disposed on an outer surface of said fluid chamber and each of which has a pair of electrodes sandwiching a piezoelectric body, and a power supply for supplying a voltage to said pair of electrodes.

7. A fluid actuator according to claim 6, wherein said piezoelectric elements are disposed on a surface of said fluid chamber to which said connecting channel is connected, and a surface opposed to said surface.

8. A fluid actuator according to claim 1, wherein said pressure generating section includes a conductive polymer actuator attached to an outer surface of said fluid chamber, and a power supply for supplying a voltage to said conductive polymer actuator.

9. A fluid actuator according to claim 8, wherein said conductive polymer actuator is disposed on a surf ace opposed to a surface of said fluid chamber to which said Connecting channel is connected.

10. A fluid actuator according to claim 8, wherein said conductive polymer actuator is disposed on approximately entire surf ace of said fluid chamber except f or an area to which said connecting channel is connected.

11. A fluid actuator according to claim 2, wherein said pressure adjusting section adjusts said pressure by changing said voltage.

12. A fluid actuator according to claim 1, wherein said plural fluid chambers have the same volume in a state of equilibrium.

13. A fluid actuator according to claim 1, wherein a cross section of each of said fluid chambers has an approximately rectangular shape.

14. A fluid actuator according to claim 1, wherein said connecting channel connects said plural fluid chambers at equal distances.

15. A fluid actuator according to claim 1, wherein said pressure generating section generates pressure between a surface of said fluid chamber to which said connecting channel is connected and a surface opposed to said surface.

16. A fluid actuator according to claim 1, wherein a surface opposed to a surface of said fluid chamber to which said connecting channel is connected is formed of said elastic member.

17. A fluid actuator according to claim 1, wherein said connecting channel is formed of a hard material which is not elastically deformed.

18. A fluid actuator according to claim 1, wherein said fluid is one of normal saline solution, water, air nitrogen, and a rare gas.

19. A fluid actuator according to claim 1, wherein said elastic member is one of silicone rubber, polyurethane rubber, and latex rubber.

20. An endoscope inserted into an object to be inspected, said endoscope having a bending section in an inserting section inserted into said object, said endoscope comprising:
a fluid actuator according to claim 1, said fluid actuator being incorporated in said bending section.

21. An endoscope according to claim 20 further including:
an operating section for operating said bending section; and
a drive control section for controlling a drive of said fluid actuator in accordance with operation of said operating section.

22. An endoscope according to claim 20, wherein a forceps channel, an air/water supplying tube, and a cable are inserted through a cavity between said fluid chamber and said connecting channel.

23. An endoscope according to claim 20, wherein said endoscope is provided with a plurality of said bending sections, and said fluid actuator is incorporated in each of said bending sections.
